(19) United States
(12) Patent Application Publication
(10) Pub. No.: US 2025/0376668 A1
(43) Pub. Date: Dec. 11, 2025

(54) CHEMICALLY MODIFIED PROTEASE ENZYMES WITH ENHANCED AUTOLYSIS RESISTANCE

(71) Applicant: Waters Technologies Corporation, Milford, MA (US)

(72) Inventors: Matthew A. Lauber, North Smithfield, RI (US); Michael Zagieboylo, Hanover, MA (US)

(21) Appl. No.: 19/229,747

(22) Filed: Jun. 5, 2025

Related U.S. Application Data
(60) Provisional application No. 63/657,430, filed on Jun. 7, 2024.

(51) Int. Cl. C12N 9/52; C12Q 1/37

(52) U.S. Cl. CPC C12N 9/52; C12Q 1/37; C12Y 304/2105

(57) ABSTRACT

The present disclosure relates to chemically modified protease enzymes, Lys-C and Lys-N, that have enhanced autolysis resistance. Also disclosed herein are methods of using such protease enzymes for improving detection of target analyte proteins in an analytical assay.

### CROSS-REFERENCE TO RELATED APPLICATIONS

This patent application is an International Application which claims priority to and the benefit of U.S. Provisional Application No. 63/657,430, filed Jun. 7, 2024, the contents of which are hereby incorporated herein by reference in their entirety.

### SEQUENCE LISTING

This patent application contains a Sequence Listing which has been submitted electronically in XML format and is hereby incorporated by reference in its entirety. The XML copy, created May 28, 2025, is named WAC-436US_SL.xml and is 6,619 bytes in size.

### BACKGROUND

Proteases are used for multiple analytical applications. For example, proteases are used in sequencing or peptide mapping of proteins as well as quality control testing of therapeutic proteins, such as monoclonal antibodies, antibody-drug conjugates (ADCs), and enzyme replacement therapies (ERTs). These analyses are frequently performed with liquid chromatography-mass spectrometry (LC-MS) instrumentation. Yet, problems exist in obtaining quality data when a protease acts on itself in via process called autolysis, which produces undesirable peptide byproducts of the protease that can obscure detection peaks of relevant protein analytes. This contaminates a sample with uninformative and disruptive peptides. Therefore, there exists a need for compositions and methods that minimize protease autolysis.

### SUMMARY OF THE DISCLOSURE

The present disclosure relates to protease enzymes with lysine cleavage specificity, such as Endopeptidase Lys-C (Lys-C) or Lys-N (Peptidyl-Lys Metalloendopeptidase), that are chemically modified (e.g., alkylated, acetylated, amidinated, or guanidinated) at lysine residues to impart enhanced autolysis resistance to the enzymes. Also disclosed are methods of using these enzymes in analytical assays, such as liquid chromatography-mass spectrometry (LC-MS), among others, for improved detection and analysis of target protein analytes.

Disclosed herein, in certain embodiments, is a protease comprising one or more chemically modified lysine residues, wherein the protease has enhanced autolysis resistance as compared to the autolysis resistance of the protease in the absence of the one or more chemically modified lysine residues, wherein the protease is a wild-type Lys-C protease. In certain embodiments, the one or more chemically modified lysine residues are at least 1, at least 2, at least 3, at least 4, at least 5, at least 6, at least 7, at least 8, at least 9, at least 10, at least 11, at least 12, at least 13, at least 14, or 15 chemically modified lysine residues. In certain embodiments, the one or more chemically modified lysine residues are at amino acid position 2, 39, 52, 54, 62, 104, 173, 178, 183, 205, 235, 254, 311, 360, and/or 408 of SEQ ID NO: 1 or amino acid position 30, 49, 106, 155, and/or 203 of SEQ ID NO: 2. In certain embodiments, the protease is from Achromabacter lyticus. In certain embodiments, the protease comprises an amino acid sequence of SEQ ID NO: 1 or SEQ ID NO: 2.

Disclosed herein, in certain embodiments, is a protease comprising one or more chemically modified lysine residues, wherein the protease has enhanced autolysis resistance as compared to the autolysis resistance of the protease in the absence of the one or more chemically modified lysine residues, wherein the protease is a wild-type Lys-N protease. In certain embodiments, the one or more chemically modified lysine residues are at least 1, at least 2, at least 3, at least 4, at least 5, at least 6, at least 7, at least 8, at least 9, at least 10, or 11 chemically modified lysine residues. In certain embodiments, the one or more chemically modified lysine residues are at position 25, 39, 53, 86, 88, 167, 283, 310, 320, and/or 329 of SEQ ID NO: 3 or amino acid position 102, 129, 139, and/or 148 of SEQ ID NO: 4. In certain embodiments, the protease is from Grifola frondosa. In certain embodiments, the protease comprises an amino acid sequence of SEQ ID NO: 3 or SEQ ID NO: 4.

In certain embodiments, the lysine residues of the protease are homogenously chemically modified. In certain embodiments, the lysine residues of the protease are homogenously and completely chemically modified. In certain embodiments, the one or more chemically modified lysine residues are modified with an alkyl moiety, acetyl moiety, amidino moiety, or guanidino moiety. In certain embodiments, the alkyl moiety is selected from the group consisting of a methyl moiety, dimethyl moiety, octanal moiety, and cyclodextrin monoaldehyde moiety. In certain embodiments, the alkyl moiety is a methyl moiety.

In certain embodiments, the protease is isolated, recombinant, or synthetic.

In certain embodiments, the autolysis resistance of the protease is enhanced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 120%, at least 140%, at least 160%, at least 180%, at least 200%, at least 250%, at least 300%, at least 350%, at least 400%, at least 450%, at least 500%, at least 600%, at least 700%, at least 800%, at least 900%, at least 1000%, or more as compared to the autolysis resistance of the protease in the absence of the one or more chemically modified lysine residues.

Disclosed herein, in certain embodiments, is a method of reducing a level of peptide byproducts of protease autolysis in an analytical assay, the method comprising the use of the protease of any one of the foregoing aspects and embodiments. In certain embodiments, the analytical assay is selected from the group consisting of liquid chromatography (LC), LC-mass spectrometry (LC-MS), LC-UV, capillary electrophoresis (CE), gel electrophoresis (GE), and matrix-assisted laser desorption/ionization (MALDI). In certain embodiments, the analytical assay is LC-MS. In certain embodiments, the method comprises the use of one or more additional protease enzymes. In certain embodiments, the one or more additional protease enzymes is a trypsin enzyme.

### DEFINITIONS

Unless defined otherwise, all technical and scientific terms used herein have the same meaning as is commonly understood by one of skill in the art to which the claimed subject matter pertains. Generally, nomenclatures utilized in connection with, and techniques of cell and tissue culture, molecular biology, and protein and polynucleotide chemistry described herein are those well-known and commonly used in the art. It is to be understood that the foregoing general description and the following detailed description are representative and explanatory only and are not restrictive of any subject matter claimed. The section headings used herein are for organizational purposes only and are not to be construed as limiting the subject matter described.

As used herein, singular forms “a,” “and,” and “the” include plural referents unless the context clearly indicates otherwise. Thus, e.g., reference to “an enzyme” includes a plurality of enzymes and reference to “an enzymes” in some embodiments includes multiple enzymes, and so forth.

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20-fold, etc., as well as 1.1, 1.2, 1.3, 1.4, 1.5-fold, etc., 2.1, 2.2, 2.3, 2.4, 2.5-fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, the phrase “analytical assay” refers to any known assay used in the relevant art for the analysis of proteins. Non-limiting examples of analytical assays include those that are used for extraction, isolation, detection, sequence analysis, structure analysis, post-translational modification analysis, and assessment of the function of a target protein ('target analyte'), among others. Specific examples of analytical assays include liquid chromatography (LC), LC-mass spectrometry (LC-MS), LC-UV, capillary electrophoresis (CE), gel electrophoresis (GE), matrix-assisted laser desorption/ionization (MALDI), hydrogen-deuterium exchange, protein sequencing, peptide mapping by electrophoresis, western blotting, protein nuclear magnetic resonance (NMR), protein footprinting, affinity purification, protein conformational studies, and proteomics, among others.

As used herein, the phrase “autolysis resistance” or variants thereof refers to a property of a protease enzyme described herein (e.g., Lys-C or Lys-N) to resist proteolytic cleavage via its own active site (i.e., self-cleavage). Protease enzymes are themselves proteins containing amino acid residues that can act as substrate residues for the protease's active site. Accordingly, the chemical modification (e.g., alkylation, acetylation, amidination, or guanidination) of these residues can introduce derivative forms that are not natural substrates for the enzyme's active site and thereby enhance the resistance of the protease to self-cleavage.

A protease enzyme disclosed herein can contain any number of amino acid residues that act as substrates for the enzyme's protease domain (e.g., 1, 2, 3, 4, 5, 6, 7, 8, 9, 10, or more amino acid residues). Accordingly, varying degrees of autolysis resistance can be conferred to the protease. For example, autolysis resistance may be conferred to the protease or enhanced by 1, 2, 3, 4, 5, 6, 7, 8, 9, 10 or more chemical modifications. The enhancement in autolysis resistance can be by any amount, including, e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 120%, at least 140%, at least 160%, at least 180%, at least 200%, at least 250%, at least 300%, at least 350%, at least 400%, at least 450%, at least 500%, at least 600%, at least 700%, at least 800%, at least 900%, or at least 1000% as compared to the autolysis resistance of the protease in the absence of the one or more chemical modifications. In certain embodiments, the degree of protease autolysis resistance is proportional to the extent of chemical modification of lysine residues of the protease (i.e., more chemically modified lysine residues result in greater autolysis resistance). In certain embodiments, chemical modification of any particular lysine residue of the protease does not modify the selectivity or cleavage of any other lysine residue of the protease.

As used herein, the phrase “chemical modification” refers to any process by which a molecule or macromolecule can be converted through a chemical reaction or a series of chemical reactions. A molecule or macromolecule produced by such a process is referred to herein as “chemically modified.” Non-limiting examples of chemical modifications include addition of an alkyl moiety, acetyl moiety, amide moiety, amidino moiety, or guanidino moiety. Non-limiting examples of an alkyl moiety include a methyl moiety, dimethyl moiety, octanal, and cyclodextrin monoaldehyde moiety.

As used herein, the phrase “homogenously chemically modified” refers to a protease enzyme of the present disclosure having 95+5% (e.g., 90%, 91%, 92%, 93%, 94%, 95%, 96%, 97%, 98%, 99%, or 100%) of its lysine residues chemically modified (e.g., alkylated, acetylated, amidinated, or guanidinated).

As used herein, the phrase “homogenously and completely chemically modified” refers to a protease enzyme of the present disclosure having between 80% and 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of its lysine residues chemically modified (e.g., alkylated, acetylated, amidinated, or guanidinated).

As used herein, the term “isolated” refers to a molecule, e.g., a protease enzyme of the present disclosure, which is purified from an organism in which it naturally occurs or from an artificial expression system (e.g., cell-free or cell-based expression system).

As used herein, the phrase “peptide byproducts of protease autolysis” refers to fragments of a protease (e.g., Lys-C or Lys-N) produced by way of autolysis by the protease. Peptide byproducts of protease autolysis can be of various sizes, depending on the length of the protease amino acid sequence and the number of amino acid residues contained therein that can act as substrates for proteolytic cleavage by the active site of the protease. Such peptide byproducts are generally undesirable in the context of certain protein assays (e.g., analytical assays disclosed herein) as they may produce interference and negatively impact the sensitivity and/or specificity of measurements produced by these assays. Non-limiting lengths of peptide byproducts of proteolysis include peptides having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more amino acid residues.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y)
   where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “recombinant” refers to a protein or a fragment thereof (e.g., a protease enzyme disclosed herein) that is encoded by a nucleic acid that has been cloned into an expression system capable of transcribing the gene for translation into a protein.

As used herein, the term “synthetic” refers to a protein or a fragment thereof (e.g., a protease enzyme disclosed herein) that is produced artificially using well-known methods, such as, e.g., solid phase or liquid phase synthesis.

As used herein, the term “wild-type” refers to a protein or a fragment thereof, such as a protease enzyme disclosed herein, which contains an amino acid sequence the of protein as it occurs in nature. The term “wild-type” also includes proteins with naturally-occurring sequences that are artificially or synthetically modified (e.g., chemically modified, e.g., by way of alkylation, acetylation, amidination, or guanidination) to produce a chemically modified protease enzyme that does not occur naturally.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Expression vectors suitable for use with the compositions and methods described herein contain a polynucleotide sequence as well as, e.g., additional sequence elements used for the expression of proteins and, optionally, the integration of these polynucleotide sequences into the genome of a host cell. Certain vectors that can be used for the expression one or more (e.g., 1, 2, 3, or more) recombinant protease enzymes, as described herein, include plasmids that contain regulatory sequences, such as promoter and enhancer regions, which direct gene transcription. Other useful vectors for expression of protease enzymes contain polynucleotide sequences that enhance the rate of translation of these genes or improve the stability or nuclear export of the mRNA that results from gene transcription. These sequence elements may include, e.g., 5′ and 3′ untranslated regions (UTRs), an internal ribosomal entry site (IRES), and a polyadenylation signal site in order to direct efficient transcription of the gene carried on the expression vector. The expression vectors suitable for use with the compositions and methods described herein may also contain a polynucleotide encoding a marker for selection of cells that contain such a vector.

### DETAILED DESCRIPTION

Protease autolysis creates interference and negatively impacts the sensitivity and specificity of LC-MS-based measurements. One issue encountered during peptide mapping, among others, is that protease enzymes are, in and of themselves, proteins and will self-digest ('autolyze') into peptide byproducts. Issues stemming from autolytic background peptides become more pronounced in cases where two or more proteases are used on the same sample. Digestion mixtures that are desired to have only peptide fragments from the protein analyte of interest ('the target analyte') will thus be contaminated with peptides from the protease(s) used in the sample preparation. Chromatographic peaks for these protease fragments appear during high-performance liquid chromatography (HPLC)-based separation, thus, making identification and structural characterization of the target analyte protein more difficult.

To date, there exists a need to minimize autolysis reaction byproducts in LC-MS analyses in order to reduce byproduct interference during detection of target analytes. Here, the present disclosure provides protease enzymes, such as Lys-C and Lys-N, that are chemically modified on one or more lysine residues to confer enhanced resistance to autolysis. The chemically modified protease enzymes of the disclosure do not exhibit these modifications under their naturally occurring state.

### Protease Enzymes

### Lys-C

Lys-C (30 kDa) is a bacterial serine protease which hydrolyzes peptide bonds on the carboxyl side of lysine (Lys) residues, particularly Lys residues that are followed by proline residues. This enzyme generally produces peptide fragments that are long and have lower complexity. Lys-C exhibits optimal protease activity at a pH range of 7.0-9.0 and is highly resistant to strong denaturing conditions (e.g., high concentrations of urea). Lys-C is naturally found to occur in Achromobacter lyticus (M497-1), Lysobacter spp., including, e.g., Lysobacter enzymogenes, Lysobacter antibioticus, Lysobacter sp. Root96, Lysobacter maris, as well as Shewanella spp., Aquimonas, Pseudofulvimonas, Tahibacter sp., Thalassocella spp., and Myxobacteria Strain AL-1. This protease is frequently used alone or in combination with other protease enzymes for various applications, including in-solution or in-gel protein digestion, phosphopeptide enrichment, protein mapping, peptide mass fingerprinting, mass spectrometry-based spectral matching, and proteomics.

The present disclosure provides chemically modified, wild-type Lys-C protease enzymes that exhibit enhanced autolysis resistance. The disclosed Lys-C protease enzymes can be obtained or derived from any biological source, including bacteria and/or artificial expression or synthesis systems. In certain embodiments, the Lys-C protease is isolated. In certain embodiments, the Lys-C protease is recombinant. In certain embodiments, the Lys-C protease is synthetic. In certain embodiments, the Lys-C protease is obtained or derived from Achromobacter lyticus. In certain embodiments, the Lys C-protease is obtained or derived from a Lysobacter spp. In certain embodiments, the Lysobacter spp. is selected from the group consisting of Lysobacter enzymogenes, Lysobacter antibioticus, Lysobacter sp. Root96, and Lysobacter maris. In certain embodiments, the Lys-C protease is obtained or derived from Myxobacteria Strain AL-1. In certain embodiments, the Lys-C protease is obtained or derived from a Shewanella spp. In certain embodiments, the Lys-C protease is obtained or derived from Aquimonas. In certain embodiments, the Lys-C protease is obtained or derived from Pseudofulvimonas. In certain embodiments, the Lys-C protease is obtained or derived from Tahibacter sp. In certain embodiments, the Lys-C protease is obtained or derived from Thalassocella spp.

The wild-type amino acid sequence of Lys-C of Achromobacter lyticus is provided in SEQ ID NO: 1, below, with bold and italicized letters demarcating lysine (“Lys” or “K”) residues at amino acid positions 2, 39, 52, 54, 62, 104, 173, 178, 183, 205, 235, 254, 311, 360, and 408 that act as natural substrates for the enzyme's proteolytic active site and which can be chemically modified (e.g., alkylated, acetylated, amidinated, or guanidinated) according to the methods of the present disclosure.

(SEQ ID NO: 1)
MKRICGSLLLLGLSISAALAAPASRPAAFDYANLSSVDKVALRTMPAVDV
AKAKAEDLQRDKRGDIPRFALAIDVDMTPQNSGAWEYTADGQFAVWRQRV
RSEKALSLNFGFTDYYMPAGGRLLVYPATQAPAGDRGLISQYDASNNNSA
RQLWTAVVPGAEAVIEAVIPRDKVGEFKLRLTKVNHDYVGFGPLARRLAA
ASGEKGVSGSCNIDVVCPEGDGRRDIIRAVGAYSKSGTLACTGSLVNNTA
NDRKMYFLTAHHCGMGTASTAASIVVYWNYQNSTCRAPNTPASGANGDGS
MSQTQSGSTVKATYATSDFTLLELNNAANPAFNLFWAGWDRRDQNYPGAI
AIHHPNVAEKRISNSTSPTSFVAWGGGAGTTHLNVQWQPSGGVTEPGSSG
SPIYSPEKRVLGQLHGGPSSCSATGTNRSDQYGRVFTSWTGGGAAASRLS
DWLDPASTGAQFIDGLDSGGGTP

The Lys-C protease includes a protease domain that performs its enzymatic function. The amino acid sequence of the wild-type Lys-C protease domain is provided in SEQ ID NO: 2, below, with bold and italicized letters demarcating Lys residues at amino acid positions 30, 49, 106, 155, and 203 that are natural substrates for the enzyme's proteolytic active site and which can be chemically modified (e.g., alkylated, acetylated, or amidinated) according to the methods of the present disclosure.

(SEQ ID NO: 2)
GVSGSCNIDVVCPEGDGRRDIIRAVGAYSKSGTLACTGSLVNNTANDRKM
YFLTAHHCGMGTASTAASIVVYWNYQNSTCRAPNTPASGANGDGSMSQTQ
SGSTVKATYATSDFTLLELNNAANPAFNLFWAGWDRRDQNYPGAIAIHHP
NVAEKRISNSTSPTSFVAWGGGAGTTHLNVQWQPSGGVTEPGSSGSPIYS
PEKRVLGQLHGGPSSCSATGTNRSDQYGRVFTSWTGGGAAASRLSDWLDP
ASTGAQFIDGLDSGGGTP

### Lys-N

Lys-N (Peptidyl-Lys Metalloendopeptidase) is a protease that specifically cleaves peptide bonds on the amino side of lysine residues. It is derived from Grifola frondosa, a type of mushroom. The wild-type amino acid sequence of Lys-N of Grifola frondosa containing its propeptide sequence is provided in SEQ ID NO: 3, below, with bold and italicized letters demarcating lysine residues at amino acid positions 25, 39, 53, 86, 88, 167, 283, 310, 320, and/or 329 that act as natural substrates for the enzyme's proteolytic active site and which can be chemically modified (e.g., alkylated, acetylated, amidinated, or guanidinated) according to the methods of the present disclosure.

(SEQ ID NO: 3)
MFSSVMVALVSLAVAVSANPGLSLKVSGPEAVDGVNNLKVVTTITNTGDE
TLKLLNDPRGALHTMPTDTFAITNESGETPSFIGVKVKYVPSMAAKSTGE
NVFAVIAPGQSVNVEHDLSAAYNFTSSGAGTYALEALNVFNYIDPETNEP
VEIWADAEAHTTAVSGKLAVVRATPTLTRPVTYNGCSSSEQSALAAAASA
AQSYVAESLSYLQTHTAATPRYTTWFGSYISSRHSTVLQHYTDMNSNDFS
SYSFDCTCTAAGTFAYVYPNRFGTVYLCGAFWKAPTTGTDSQAGTLVHES
SHFTRNGGTKDYAYGQAAAKSLATMDPDKAVMNADNHEYFSENNPAQS

The Lys-N protease includes a protease domain that performs its enzymatic function. The amino acid sequence of the wild-type Lys-N protease domain, corresponding to residues 182 to 348 of the full-length wild-type sequence, is provided in SEQ ID NO: 4, below, with bold and italicized letters demarcating Lys residues at amino acid positions 102, 129, 139, and/or 148 that are natural substrates for the enzyme's proteolytic active site and which can be chemically modified (e.g., alkylated, acetylated, or amidinated) according to the methods of the present disclosure.

(SEQ ID NO: 4)
TYNGCSSSEQSALAAAASAAQSYVAESLSYLQTHTAATPRYTTWFGSYIS
SRHSTVLQHYTDMNSNDFSSYSFDCTCTAAGTFAYVYPNRFGTVYLCGAF
WKAPTTGTDSQAGTLVHESSHFTRNGGTKDYAYGQAAAKSLATMDPDKAV
MNADNHEYFSENNPAQS

The present disclosure further provides chemically modified, wild-type Lys-N protease enzymes that exhibit enhanced autolysis resistance. The disclosed Lys-N protease enzymes can be obtained or derived from any biological source, including bacteria and/or artificial expression or synthesis systems. In certain embodiments, the Lys-N protease is isolated. In certain embodiments, the Lys-N protease is recombinant. In certain embodiments, the Lys-N protease is synthetic. In certain embodiments, the Lys-C protease is obtained or derived from Grifola frondosa.

The unique specificity of Lys-N makes it particularly useful in proteomics and protein sequencing applications because it generates peptides with a positively charged lysine at one end, which can be advantageous for certain mass spectrometry (MS) analyses. This protease is often used in bottom-up proteomics approaches in which proteins are enzymatically digested into peptides before MS analysis. The predictable cleavage pattern of Lys-N can simplify the analysis and interpretation of MS data, aiding in the identification and quantification of proteins. In comparison to trypsin, another commonly used protease in proteomics that also cleaves at lysine residues, Lys-N can generate different peptide fragments, potentially providing complementary information. This can be particularly useful for improving protein coverage or for analyzing proteins that may be difficult to digest or sequence using trypsin alone. Due to its specific cleavage pattern and the fact that it works well under conditions that are not ideal for some other proteases (e.g., acidic conditions), Lys-N has become an important tool in the toolkit of researchers conducting proteomics studies.

### Chemical Modifications

Autolysis resistance of lysine-specific protease enzymes disclosed herein (e.g., Lys-C and Lys-N) can be enhanced by the addition of a chemical modification at one or more (e.g., 1, 2, 3, 4, 5, 6, 7, 8, 9, 10, or more) amino acid residues that are natural substrates for the enzyme's active site. In certain embodiments, the chemical modification is at one or more lysine residues of the protease (e.g., Lys-C or Lys-N). In certain embodiments, the chemical modification comprises an addition of a chemical moiety on one or more lysine residues (e.g., 1, 2, 3, 4, 5, 6, 7, 8, 9, 10, or more) selected from the group consisting of an alkyl moiety, acetyl moiety, amidino moiety, and guanidino moiety.

Accordingly, the present disclosure provides compositions and methods for chemical modification (e.g., alkylation, acetylation, amidination, and guanidination) of proteases of the disclosure (e.g., Lys-C and Lys-N) in order to enhance autolysis resistance of the enzymes.

In certain embodiments, the chemical modification is alkylation of a lysine residue of a protease enzyme disclosed herein (e.g., Lys-C or Lys-N). In certain embodiments, the alkyl group is attached to an amine group of one or more lysine residues of the enzyme. In certain embodiments, the alkyl group is a primary or branched C₁₋₁₂ alkyl group. In certain embodiments, chemically modified proteases of the present disclosure are those in which the alkyl group is a primary or branched C₁₋₄ alkyl group. Alkylation of protease enzymes is generally performed by reductive alkylation. The degree of alkylation of amino acid residues will depend on the reaction conditions of the reductive alkylation process. For example, if the reaction cycle is repeated a number of times and/or a higher reagent: enzyme ratio is used, then full alkylation, i.e., alkylation of all target residues will be achieved. In certain embodiments, chemically modified protease enzymes of the present disclosure may be fully di-alkylated at all of their target amino acid (e.g., Lys) residues. In certain embodiments, chemically modified protease enzymes of the present disclosure may be partially alkylated at one or more (e.g., 1, 2, 3, 4, 5, 6, 7, 8, 9, 10, or more) of their target amino acid (e.g., Lys) residues. In certain embodiments, the lysine residues of the protease are homogenously alkylated (i.e., 95±5% of lysine residues of the protease are alkylated). In certain embodiments, the lysine residues of the protease are homogenously and completely alkylated (i.e., 80%- 100% of lysine residues of the protease are alkylated). In certain embodiments, the alkyl moiety is selected from the group consisting of a methyl moiety, dimethyl moiety, octanal moiety, and cyclodextrin monoaldehyde moiety. In certain embodiments, the alkylating moiety comprises a polyethylene glycol chain or is a bifunctional reagent capable of intramolecularly crosslinking two lysine residues. A representative, non-limiting method for reductive methylation of a protease is described herein in Example 2.

In certain embodiments, the chemical modification is acetylation of a lysine residue of a protease enzyme disclosed herein (e.g., Lys-C or Lys-N). In certain embodiments, the acetyl group is attached to an amine group of an amino acid residue (e.g., lysine) of the enzyme. Acetylation of protease enzymes can be performed using known methods, for example, by derivatization with Sulfo-NHS-Acetate. The degree of acetylation of amino acid residues will depend on the reaction conditions of the derivatization process. For example, if the reaction cycle is repeated a number of times and/or a higher reagent: enzyme ratio is used, then full acetylation, i.e., acetylation of all target residues will be achieved. In certain embodiments, chemically modified protease enzymes of the present disclosure may be fully acetylated at all of their target amino acid (e.g., Lys) residues. In certain embodiments, chemically modified protease enzymes of the present disclosure may be partially acetylated at one or more (e.g., 1, 2, 3, 4, 5, 6, 7, 8, 9, 10, or more) of their target amino acid (e.g., Lys) residues. In certain embodiments, the lysine residues of the protease are homogenously acetylated (i.e., 95±5% of lysine residues of the protease are acetylated). In certain embodiments, the lysine residues of the protease are homogenously and completely acetylated (i.e., 80%- 100% of lysine residues of the protease are acetylated). A representative, non-limiting method for acetylation of a protease is described herein in Example 3.

In certain embodiments, the chemical modification is amidination of a lysine residue of a protease enzyme disclosed herein (e.g., Lys-C or Lys-N). In certain embodiments, the amidino group is attached to an amine group of an amino acid residue (e.g., lysine) of the enzyme. Amidination of protease enzymes can be performed using known methods, for example, by derivatization with S-methyl thioacetamide. The degree of amidination of amino acid residues will depend on the reaction conditions of the derivatization process. For example, if the reaction cycle is repeated a number of times and/or a higher reagent: enzyme ratio is used, then full amidination, i.e., amidination of all target residues will be achieved. In certain embodiments, chemically modified protease enzymes of the present disclosure may be fully amidinated at all of their target amino acid (e.g., Lys) residues. In certain embodiments, chemically modified protease enzymes of the present disclosure may be partially amidinated at one or more (e.g., 1, 2, 3, 4, 5, 6, 7, 8, 9, 10, or more) of their target amino acid (e.g., Lys) residues. In certain embodiments, the lysine residues of the protease are homogenously amidinated (i.e., 95+5% of lysine residues of the protease are amidinated). In certain embodiments, the lysine residues of the protease are homogenously and completely amidinated (i.e., 80%- 100% of lysine residues of the protease are amidinated). A representative, non-limiting method for amidination of a protease is described herein in Example 4.

In certain embodiments, the chemical modification is guanidination of a lysine residue of a protease enzyme disclosed herein (e.g., Lys-C or Lys-N). In certain embodiments, the guanidine group is attached to an amine group of an amino acid residue (e.g., lysine) of the enzyme. Guanidination of protease enzymes can be performed using known methods, for example, by derivatization with O-Methylisourea bisulfate. The degree of guanidination of amino acid residues will depend on the reaction conditions of the derivatization process. For example, if the reaction cycle is repeated a number of times and/or a higher reagent: enzyme ratio is used, then full guanidination, i.e., guanidination of all target residues will be achieved. In certain embodiments, chemically modified protease enzymes of the present disclosure may be fully guanidinated at all of their target amino acid (e.g., Lys) residues. In certain embodiments, chemically modified protease enzymes of the present disclosure may be partially guanidinated at one or more (e.g., 1, 2, 3, 4, 5, 6, 7, 8, 9, 10, or more) of their target amino acid (e.g., Lys) residues. In certain embodiments, the lysine residues of the protease are homogenously guanidinated (i.e., 95±5% of lysine residues of the protease are guanidinated). In certain embodiments, the lysine residues of the protease are homogenously and completely guanidinated (i.e., 80%- 100% of lysine residues of the protease are guanidinated). A representative, non-limiting method for guanidination of a protease is described herein in Example 5.

It may be desirable, in certain embodiments, to chemically modify certain target lysine residues within a protease enzyme of the disclosure, while preventing chemical modification of other non-target lysine residues within the enzyme (e.g., lysine residues within the active site of the protease). In such cases, one may prevent modification particular lysine residues by performing the chemical modification in the presence of a non-covalent (i.e., reversible) inhibitor that binds to the non-target residues. Without wishing to be bound by any particular theory, a non-covalent inhibitor may reversibly bind to a non-target lysine residue for which chemical modification is undesirable, thereby sterically hindering addition of a covalently modifying moiety to the non-target lysine residue. Nonlimiting examples of such non-covalent inhibitors include aprotinin and leupeptin.

Disclosed herein, in certain embodiments, is a wild-type Lys-C enzyme that is chemically modified at one or more (e.g., 1, 2, 3, 4, 5, 6, 7, 8, 9, 10, 11, 12, 13, 14, 15, or more) amino acid residues. In certain embodiments, the wild-type Lys-C enzyme is chemically modified at one or more lysine residues. In certain embodiments, the one or more chemically modified lysine residues are at least 1, at least 2, at least 3, at least 4, at least 5, at least 6, at least 7, at least 8, at least 9, at least 10, at least 11, at least 12, at least 13, at least 14, or 15 chemically modified lysine residues. In certain embodiments, the one or more chemically modified lysine residues of the Lys-C protease are at amino acid position 2, 39, 52, 54, 62, 104, 173, 178, 183, 205, 235, 254, 311, 360, and/or 408 of SEQ ID NO: 1 or amino acid position 30, 49, 106, 155, and/or 203 of SEQ ID NO: 2. In certain embodiments, the Lys-C protease is from Achromabacter lyticus. In certain embodiments, the Lys-C protease comprises an amino acid sequence of SEQ ID NO: 1 or SEQ ID NO: 2. In certain embodiments, the chemically modified Lys-C protease has enhanced autolysis resistance as compared to the autolysis resistance of the Lys-C protease in the absence of the one or more chemically modified lysine residues. In certain embodiments, the wild-type Lys-C enzyme is modified to incorporate one or more amino acid substitutions. In certain embodiments, the one or more amino acid substitutions are one or more conservative amino acid substitutions. In certain embodiments, the one or more conservative amino acid substitutions is a lysine (Lys) to arginine (Arg) substitution.

Disclosed herein, in certain embodiments, is a wild-type Lys-N enzyme that is chemically modified at one or more (e.g., 1, 2, 3, 4, 5, 6, 7, 8, 9, 10, 11, or more) amino acid residues. In certain embodiments, the wild-type Lys-N enzyme is chemically modified at one or more lysine residues. In certain embodiments, the one or more chemically modified lysine residues are at least 1, at least 2, at least 3, at least 4, at least 5, at least 6, at least 7, at least 8, at least 9, at least 10, or 11, chemically modified lysine residues. In certain embodiments, the one or more chemically modified lysine residues of the Lys-N protease are at amino acid position 25, 39, 53, 86, 88, 167, 283, 310, 320, and/or 329 of SEQ ID NO: 3 or amino acid position 102, 129, 139, and/or 148 of SEQ ID NO: 4. In certain embodiments, the protease is from Grifola frondosa. In certain embodiments, the protease comprises an amino acid sequence of SEQ ID NO: 3 or SEQ ID NO: 4. In certain embodiments, the chemically modified Lys-N protease has enhanced autolysis resistance as compared to the autolysis resistance of the Lys-N protease in the absence of the one or more chemically modified lysine residues. In certain embodiments, the wild-type Lys-N enzyme is modified to incorporate one or more amino acid substitutions. In certain embodiments, the one or more amino acid substitutions are one or more conservative amino acid substitutions. In certain embodiments, the one or more conservative amino acid substitutions is a lysine (Lys) to arginine (Arg) substitution.

### Protease Purification

The disclosed protease enzymes can be isolated from a target organism in which it is naturally produced or from an artificial or recombinant expression system subsequent to cell-based or cell-free expression using a variety of well-known protein purification methods.

Purification generally begins with preparation of a crude extract containing a complex mixture of all proteins from the cell cytoplasm and various other macromolecules, cofactors, and nutrients. Crude extracts are prepared, In certain embodiments, using chemical methods, enzymatic methods, sonication, or a French press. Subsequently, debris may be removed from the crude extract by centrifugation, and the supernatant containing the expressed proteases is retrieved. Various well-known methods may then be used to isolate the protein from the supernatant, including but not limited to, chromatographic methods (e.g., affinity chromatography, HPLC, SEC, and IEC), protein precipitation, and cation exchange and gel filtration. Confirmation of protein purity may be performed by well-known methods, including, e.g., HPLC, MS, SDS-PAGE, ELISA, Bradford assay, ultraviolet-visible spectroscopy, activity assays, dynamic light scattering, microfluidic diffusional sizing, sedimentation velocity methods, and immunoblotting.

### Recombinant Protease Expression

The protease enzymes disclosed herein (e.g., Lys-C, Lys-N or other) may be produced using a recombinant expression system. For example, a polynucleotide (e.g., DNA or RNA) encoding a protease of the disclosure may be incorporated into a recombinant expression vector capable of supporting and facilitating the expression of the protease in a host cell.

In a non-limiting example, disclosed herein are methods for expressing a wild-type protease of the disclosure using a recombinant expression system, including: (1) transforming a host cell with a recombinant nucleic acid comprising a sequence which encodes the protease from an bacterial, fungal, plant, or mammalian source; and (2) culturing the host cell under conditions and for a time sufficient to allow for the stable expression of the protease; and (3) isolating the expression product from the culture medium.

Representative methods that can be used for effectuating the expression of one or more proteases of the disclosure in a host cell are described in further detail below. One platform that can be used to achieve effective intracellular concentrations of one or more proteases described herein in host cells is via stable expression of genes encoding these enzymes (e.g., by integration into the nuclear or mitochondrial genome of a host cell). These genes are polynucleotides that encode the primary amino acid sequence of the corresponding protein. In order to introduce such exogenous genes into a host cell, these genes can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are conventional and well-known.

Genes encoding enzymes of the disclosure can also be introduced into host cells by targeting a vector containing a gene encoding such an enzyme to cell membrane phospholipids.

Recognition and binding of the polynucleotide encoding a recombinant protein by RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a promoter, the sequence of which can be recognized and bound by specific transcription initiation factors and ultimately RNA polymerase, and which is operably linked to (e.g., is upstream of) the protease coding sequence. General examples of promoter classes suitable for use with the disclosed compositions and methods include constitutive promoters, spatiotemporal promoters, inducible promoters, and synthetic promoters. Additional regulatory elements such as enhancers, terminators, and the like may also be used to direct the transcription of a nucleic acid encoding one or more protease enzymes of the disclosure.

Once a polynucleotide encoding one or more proteases of the disclosure has been internalized by the host cell extrachromosomally and/or incorporated into the nuclear DNA of the host cell, the transcription of this polynucleotide can be induced by methods known in the art.

In certain embodiments, it may be desirable to express a protease of the present disclosure (e.g., Lys-C and Lys-N) as a fusion protein, e.g., to increase protease solubility, facilitate purification, and/or expression yield. In certain embodiments, a protease of the disclosure is expressed as a fusion protein comprising the protease and a second protein. In certain embodiments, the protease of the disclosure is expressed as a fusion protein comprising the protease domain (e.g., SEQ ID NO: 2 or SEQ ID NO: 4) and a second protein. In certain embodiments, the second protein is a maltose-binding protein (MBP) domain, His-tag (e.g., 6x-His [SEQ ID NO: 5] ), maltose binding protein tag, SNAP tag, FLAG tag, halotag, or fluorescent protein tag.

### Expression Vectors

A variety of vectors for the delivery of polynucleotides encoding exogenous proteins to a host cell have been developed. Expression vectors for use in the compositions and methods described herein may contain one or more polynucleotides encoding one or more protease enzymes of the disclosure, and may further include, for example, one or more (e.g., 1, 2, 3, 4, 5, 6, 7, 8, 9, 10, or more) nucleic acid elements used to regulate the expression of these agents and/or the integration of such polynucleotides into the genome of a host cell.

In certain embodiments, the vector may be an autonomously replicating vector, i.e., a vector which exists as an extrachromosomal entity, the replication of which is independent of chromosomal replication, e.g., a plasmid, viral vector, extrachromosomal element, mini-chromosome, or an artificial chromosome. Alternatively, the vector may be one which, when introduced into a host cell, is integrated into the host cell genome and replicated together with the chromosome(s) into which it has been integrated. Certain vectors that can be used for the expression of one or more engineered proteases described herein include plasmids that contain regulatory sequences, such as promoter and, optionally, enhancer regions, which direct gene transcription. Other useful vectors for expression of one or more protease enzymes of the disclosure contain polynucleotide sequences that enhance the rate of translation of these genes or improve the stability or nuclear export of the mRNA that results from gene transcription. These sequence elements include, e.g., 5′ and 3′ untranslated regions, an internal ribosome entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. The aforementioned vectors are conventional and well-known in the art.

In certain embodiments, expression vectors of the present disclosure further include a polynucleotide encoding a protein tag, such as, a His-tag (e.g., 6x-His [SEQ ID NO: 5] ), maltose binding protein tag, SNAP tag, FLAG tag, halotag, fluorescent protein tag, and the like.

### Assays for Assessing Autolysis and Target Proteolysis

The present disclosure further provides assays that are suitable for assessing the autolysis resistance of protease enzymes disclosed herein as well as the digestion efficiency of target proteins ('target proteolysis'). In certain embodiments, autolysis resistance of the chemically modified proteases of the disclosure is assessed by way of HPLC. In certain embodiments, autolysis resistance of the chemically modified proteases of the disclosure is assessed by way of MS. In certain embodiments, autolysis resistance of the chemically modified proteases of the disclosure is assessed by way of size exclusion chromatography (SEC). In certain embodiments, autolysis resistance of the chemically modified proteases of the disclosure is assessed by way of HPLC, MS, SEC, HPLC-UV, or any combination thereof. A representative, non-limiting method for testing protease autolysis resistance is described herein in Example 6.

Certain combinations of chemical modifications to any one of the modified protease enzymes disclosed herein may produce unexpected effects on the proteolytic activity of said enzymes. Accordingly, the present disclosure provides methods for assaying the impact of disclosed chemical modifications on the protease's enzymatic activity on a target protein. In certain embodiments, proteolytic efficacy of the chemically modified proteases of the disclosure on a target protein is assessed by way of MS. In certain embodiments, proteolytic efficacy of the chemically modified proteases of the disclosure on a target protein is assessed by way of size exclusion chromatography (SEC). In certain embodiments, proteolytic efficacy of the chemically modified proteases of the disclosure on a target protein is assessed by way of HPLC, MS, SEC, HPLC-UV, or any combination thereof. A representative, non-limiting method for testing proteolytic efficacy of a protease is described herein in Example 7.

### Assays for Assessing Extent of Chemical Modification of Protease Enzymes

The present disclosure further methods for determining the extent of chemical modification of the disclosed protease enzymes (e.g., Lys-C-and Lys-N). For example, selective chemical modification of lysine residues of the disclosed protease enzymes can be performed using mass spectrometric methods described in Chang et al. Anal. Chem. 83(23):9092-99 (2011) and Lauber et al. J. Proteome Res. 8 (9): 4193-4206 (2009), the disclosures of which are incorporated herein in their entireties.

### Methods of Use

The present disclosure provides methods for using the disclosed chemically modified proteases in a variety of uses. As discussed above, protease enzymes having enhanced autolysis resistance are particularly useful for analytical methods for analyzing proteins, including HPLC and/or MS. Autolysis produces undesirable peptide fragments from the protease itself during target analyte proteolysis, resulting in interference peaks that appear during HPLC or MS separation, thereby obfuscating peptide peaks corresponding to the analyte of interest. Thus, an autolysis-resistant protease advantageously minimized such interference peaks and improves the sensitivity and specificity of HPLC and/or MS measurements.

Furthermore, the disclosed autolysis resistant protease enzymes are well-suited for use in a variety of other applications, including HPLC-UV, development of cell and tissue culture protocols, protein degradation, protein sequencing, peptide mapping, dissociation of adherent cells, analysis of protein-protein interactions, capillary electrophoresis (CE), gel electrophoresis (GE), matrix-assisted laser desorption/ionization (MALDI), hydrogen-deuterium exchange, peptide mapping by electrophoresis, western blotting, protein nuclear magnetic resonance (NMR), protein footprinting, affinity purification, protein imaging, proteomic analysis, and protein conformational studies.

Additionally, the disclosed protease enzymes may be used in conjunction with methods for digestion and analysis of protein therapeutics, viral vector proteomes, and protein compositions of T cell and CAR-T cell therapies. For example, recombinant proteins are frequently used in biotherapeutic applications and are typically characterized for their properties and modifications (e.g., purity, amino acid sequence, post-translational modifications, mutations, etc.) using MS as well as other techniques, including HPLC, SEC, and HPLC-UV. As discussed herein, such analytic techniques are highly sensitive to byproducts of autolysis and would, therefore, benefit from use of chemically modified, autolysis-resistant protease enzymes that minimize contamination of the analyte sample with irrelevant and disruptive peptide peaks.

### Kits

The compositions described herein can be provided in a kit for use in any practical application described herein. The compositions may include one or more of the chemically modified protease enzymes disclosed herein in a suitable container means. In certain embodiments, the container means is any suitable container which houses, e.g., a liquid or lyophilized composition including, but not limited to, a vial, test tube, ampoule, bottle, or syringe. A syringe holds any volume of liquid suitable for injection into a subject, including, but not limited to, 0.5 cc, 1 cc, 2 cc, 5 cc, 10 cc, or more. In certain embodiments, such containers include injection and/or blow-molded plastic containers into which the desired vials are retained. In certain embodiments, kits also include printed material for use of the materials in the kit. In certain embodiments, such containers include injection and/or blow-molded plastic containers into which the desired vials are retained. In certain embodiments, kits also include printed material for use of the materials in the kit. Additionally, in certain embodiments, the preparations contain stabilizers to increase the shelf-life of the kits and include, e.g., bovine serum albumin (BSA). Where the compositions are lyophilized, the kit contains, In certain embodiments, further preparations of solutions to reconstitute the lyophilized preparations. Acceptable reconstitution solutions are well known in the art and include, e.g., phosphate buffered saline (PBS).

The term “packaging material” refers to a physical structure housing the components of the kit. In certain embodiments, the packaging material maintains the components sterile and is made of material commonly used for such purposes (e.g., paper, corrugated fiber, glass, plastic, foil, ampules, etc.). In certain embodiments, the label or packaging insert includes appropriate written instructions (e.g., instructing the user of the kit to perform one or more methods disclosed herein). Kits, in certain embodiments, additionally include labels or instructions for using the kit components in any method of the disclosure. In certain embodiments, a kit includes a compound in a pack or dispenser together with instructions for administering the compound in a method described herein. The instructions are, in certain embodiments, on “printed matter,” e.g., on paper or cardboard within or affixed to the kit, or on a label affixed to the kit or packaging material, or attached to a vial or tube containing a component of the kit. Instructions are additionally included on a computer readable medium, such as, e.g., CD-ROMs, DVDs, flash memory devices, solid state memory, magnetic disks and disk devices, magnetic tapes, cloud computing systems and services, and the like, In certain embodiments. In some cases, the program and instructions are permanently, substantially permanently, semi-permanently, or non-transitorily encoded on the media.

### EXAMPLES

The following examples are put forth to provide those of ordinary skill in the art with a description of how the compositions and methods described herein may be used, made, and evaluated, and are intended to be purely representative of the disclosure and are not intended to limit the scope of what the inventors regard as their invention.

### Example 1: Vector Transformation and Recombinant Protein Expression and Purification

Nucleotide sequence encoding each of Lys-C (e.g., SEQ ID NO: 1 or SEQ ID NO: 2) or Lys-N (e.g., SEQ ID NO: 3 or SEQ ID NO: 4), are cloned into protein expression vectors such as pET20 or pET21b that contain affinity tags (e.g., His-tag) through standard molecular cloning procedures. The recombinant plasmids are verified for their sequence accuracy through DNA sequencing methods and mobilized into a suitable strain of E. coli through electroporation or chemical based transformation. The recombinant protein is induced for its expression at an appropriate growth stage of the bacterial host. The expressed protein is purified by affinity chromatography and analyzed for its quality by denaturing polyacrylamide gels (SDS-PAGE) and/or liquid chromatography-mass spectrometry (LC-MS). The purified proteins are subsequently chemically modified according to the disclosed methods.

### Example 2: Alkylation of Protease Enzymes

Following purification of the protease (e.g., Lys-C or Lys-N), the purified enzymes are methylated at lysine residues to improve autolysis resistance. The protease protein is diluted with triethylammonium bicarbonate buffer (pH 8.5; 50 mM) to 1 mg/mL and treated with 2.2 μL of 36% formaldehyde, and 20 μL of sodium cyanoborohydride (NaBH₃CN; 0.6 M) per mg of trypsin for 10 minutes at room temperature. This reaction procedure is, optionally, carried at higher pH to minimize autolysis during the alkylation procedure. Optionally, the reaction is also carried out in the presence of a non-covalent inhibitor.

Following reductive methylation, the protease can be optionally purified using conventional methods. For example, in embodiments where the protease is recombinantly expressed as a fusion protein comprising a protein tag (e.g., His-tag, HA-tag, GST-tag, FLAG-tag), an affinity chromatography column with a stationary phase specific for the tag may be used. Purification may alternatively be performed using FPLC and ion exchange chromatography. As a further alternative, cold acetone precipitation and centrifugation are applied to purify the protease. Extent of derivatization is measured by LC-MS.

### Example 3: Acetylation of Protease Enzymes

Following purification of the protease (e.g., Lys-C or Lys-N), the purified enzymes are acetylated at lysine residues to improve autolysis resistance. A 1 mL solution of 100 mM HEPES pH 8 buffered 1 mg/mL protease was mixed with 50 microliters of 40 mg/mL Sulfo-NHS-Acetate dissolved in anhydrous dimethylsulfoxide (DMSO). This mixture is allowed to stand at room temperature for 1 h. The modified protease is then precipitated by cold acetone precipitation. Alternatively, the modified protease is desalted with a cross-linked dextran gel filtration desalting column or buffer exchanged with a 10K molecular weight cut-off filter. The derivatization procedure is optionally be performed in the presence of a non-covalent protease inhibitor.

Following acetylation, the protease can be optionally purified by using conventional methods. For example, in embodiments where the protease is recombinantly expressed as a fusion protein comprising a protein tag (e.g., His-tag, HA-tag, GST-tag, FLAG-tag), an affinity chromatography column with a stationary phase specific for the tag may be used. Purification may alternatively be performed using FPLC and ion exchange chromatography. As a further, cold acetone precipitation and centrifugation are applied to purify the protease. Extent of derivatization is measured by LC-MS.

### Example 4: Amidination of Protease Enzymes

Following purification of the protease (e.g., Lys-C or Lys-N), the purified enzymes are amidinated at lysine residues to improve autolysis resistance. A 1 mL solution of 100 mM HEPES pH 8 buffered 1 mg/mL protease is mixed with 50 microliters of 20 mg/ml S-methyl thioacetamide dissolved in anhydrous DMSO. This mixture is allowed to stand at room temperature for 1 h. The modified protease is then precipitated by cold acetone precipitation. Alternatively, the modified protease is desalted with a cross-linked dextran gel filtration desalting column or buffer exchanged with a 10K molecular weight cut-off filter. The derivatization procedure is optionally be performed in the presence of a non-covalent protease inhibitor.

Following amidination, the protease can be optionally purified by using conventional methods. For example, in embodiments where the protease is recombinantly expressed as a fusion protein comprising a protein tag (e.g., His-tag, HA-tag, GST-tag, FLAG-tag), an affinity chromatography column with a stationary phase specific for the tag may be used. Purification may alternatively be performed using FPLC and ion exchange chromatography. As a further, cold acetone precipitation and centrifugation are applied to purify the protease. Extent of derivatization is measured by LC-MS.

### Example 5: Guanidination of Protease Enzymes

Following purification of the protease (e.g., Lys-C or Lys-N), the purified enzymes are guanidinated at lysine residues to improve autolysis resistance. A 1 mL solution of 100 mM HEPES pH 8 buffered 1 mg/mL protease is mixed with 50 microliters of 30 mg/mL O-Methylisourea bisulfate dissolved in anhydrous DMSO. This mixture is allowed to stand at room temperature for 1 h. The modified protease is then precipitated by cold acetone precipitation. Alternatively, the modified protease was desalted with a cross-linked dextran gel filtration desalting column or buffer exchanged with a 10K molecular weight cut-off filter. The derivatization procedure is optionally be performed in the presence of a non-covalent protease inhibitor.

Following guanidination, the protease can be optionally purified by using conventional methods. For example, in embodiments where the protease is recombinantly expressed as a fusion protein comprising a protein tag (e.g., His-tag, HA-tag, GST-tag, FLAG-tag), an affinity chromatography column with a stationary phase specific for the tag may be used. Purification may alternatively be performed using FPLC and ion exchange chromatography. As a further, cold acetone precipitation and centrifugation are applied to purify the protease. Extent of derivatization is measured by LC-MS.

### Example 6: Assay for Measuring Protease Autolysis

Autodigestion of a chemically modified protease disclosed herein is monitored by incubating defined amounts of the protease in 50 mM ammonium bicarbonate buffer at 37° C. for 10 minutes to 16 hours and analyzing the resulting solution by reverse-phase high-performance liquid chromatography (RP-HPLC) using gradient chromatography. Mobile phases for this analysis include 0.1% trifluoroacetic acid (TFA) acidified water (A) and acetonitrile (B). Difluoroacetic acid or formic acid is also applied as a mobile phase additive along with other types of RP columns. The percentage of intact protein and autolytic peptides is evaluated by LC-UV or LC-MS analysis. Alternatively, SEC can be applied to assay intact versus autolyzed protease.

### Example 7: Assay to Test the Digestion Efficiency of a Protease

A target protein, such as NIST mAb reference material 8671 or a small protein (e.g., a lysozyme or cytochrome C) (10 μg in 10 μL) is denatured with 6 M guanidinium hydrochloride (90 μL) and treated with dithiothreitol (2 μL; 250 mM) for 30 minutes at room temperature to reduce the disulfide bonds. The reduced cysteines are then derivatized by iodoacetamide (3 μL; 350 mM) in dark for 30 minutes at room temperature to impede the subsequent re-formation of disulfide bonds. Subsequently, the protein is optionally desalted on a gel filtration by gravity or spin column using digestion buffer (100 mM Tris pH 7.5), concentration adjusted to 0.2 μg/μL, and digested with protease (1:5 or 1:20 ratio) for 60 minutes at 37° C. The reaction is quenched with 1% formic acid (20 μL) and stored at-20° C. for subsequent LC-MS analysis.

Liquid chromatography is performed with a conventional LC column (130 Å, 1.7 μm, 2.1×150 mm) using a 65° C. column temperature and 0.1% formic acid or 0.05% difluoroacetic acid-modified water and acetonitrile mobile phase. A 0.25 mL/min flow rate is applied. Gradient conditions are programmed with a hold at 1% B solution for 5 minutes, change to 40% B solution in 65 minutes, change to 70% B solution in 3 minutes followed by a hold of 2 minutes, then a switch to re-equilibration conditions (1% B solution), and a hold for 15 minutes.

Mass spectrometry data is acquired in full-scan mode using a time of flight mass spectrometer operating with a scan range of 50-2000 m/z at 2 Hz in positive ion mode. Electrospray source conditions are programmed for a 350° C. desolvation temperature, 20V cone voltage, and 1.2 kV capillary voltage when using an instrument containing a benchtop ToF mass spectrometer. Fragmentation data are acquired in MSe mode by ramping the cone voltage to 60V-120V range.

### Example 8: Assay for a One Pot Digestion

The chemically modified proteases disclosed herein can be used independently or in combination with another protease in a protein digestion procedure as follows. A monoclonal antibody is digested with an alkylated Achromobacter lyticus protease I (Lys-C) under nonreducing conditions. Reconstituted stocks of the antibody (21 mg/mL) are first denatured in the presence of iodoacetamide. The antibody is diluted to 2.5 mg/mL into a buffer with a final composition of 6 M GuHCl, 0.5 mM iodoacetamide, and 0.1 M phosphate (pH 7.1) and then incubated for 2 h at 37° C. Denatured protein is then diluted to 0.4 mg/mL with a urea buffer and mixed with Lys-C at an 8:1 w/w ratio. The final buffer composition during digestion is 2.9 M urea, 1.0 M GuHCl, 0.04 M hydroxylamine, and 0.08 mM iodoacetamide (pH 7.1). Lys-C digestions are incubated at 37° C. for 16 hours and then quenched by acidification with TFA and stored at 4° C.

### Example 9: Assay for In-Solution Protein Digestion for a Proteomics Sample

The chemically modified proteases disclosed herein can be used independently or in combination with a protease, such as trypsin, in an in-solution protein digestion procedure as follows. The protein sample is digested with alkylated Achromobacter lyticus Lys-C protease and trypsin protease under reducing conditions. Reconstituted stocks of the protein sample, for example, tissue, plasma, whole cell lysate, or other proteomics protein samples are first denatured in the presence of iodoacetamide. The sample is solubilized into 0.1% RapiGest SF (w:v) and 100 mM Tris-HCl (pH 7.1). Then dithiothreitol (DTT) is added to a final concentration of 5 mM and heated to 60° C. for 30 minutes prior to cooling at room temperature. Subsequently, iodoacetamide is added to a final concentration of 15 mM and incubated at room temperature for 30 minutes and then incubated for 2 h at 37° C. Denatured protein is then mixed with Lys-C and trypsin mix at 1:5 enzyme mix to protein ratio. Lys-C and trypsin mix digestions are incubated at 37° C. for 3 hours and then quenched by acidification with TFA and stored at 4° C.

### ENUMERATED EMBODIMENTS

Further embodiments contemplated by the present disclosure are enumerated below.
E1. A protease comprising one or more chemically modified lysine residues, wherein the protease has enhanced autolysis resistance as compared to the autolysis resistance of the protease in the absence of the one or more chemically modified lysine residues, wherein the protease is a wild-type Endopeptidase Lys-C (Lys-C) protease.
E2. The protease of E1, wherein the one or more chemically modified lysine residues are at least 1, at least 2, at least 3, at least 4, at least 5, at least 6, at least 7, at least 8, at least 9, at least 10, at least 11, at least 12, at least 13, at least 14, or 15 chemically modified lysine residues.
E3. The protease of El or E2, wherein the one or more chemically modified lysine residues are at amino acid position 2, 39, 52, 54, 62, 104, 173, 178, 183, 205, 235, 254, 311, 360, and/or 408 of SEQ ID NO: 1 or amino acid position 30, 49, 106, 155, and/or 203 of SEQ ID NO: 2.
E4. The protease of any one of E1-E3, wherein the protease is from Achromabacter lyticus.
E5. The protease of any one of E1-E4, wherein the protease comprises an amino acid sequence of SEQ ID NO: 1 or SEQ ID NO: 2.
E6. A protease comprising one or more chemically modified lysine residues, wherein the protease has enhanced autolysis resistance as compared to the autolysis resistance of the protease in the absence of the one or more chemically modified lysine residues, wherein the protease is a wild-type Peptidyl-Lys Metalloendopeptidase (Lys-N) protease.
E7. The protease of E6, wherein the one or more chemically modified lysine residues are at least 1, at least 2, at least 3, at least 4, at least 5, at least 6, at least 7, at least 8, at least 9, at least 10, or 11 chemically modified lysine residues.
E8. The protease of E6 or E7, wherein the one or more chemically modified lysine residues are at position 25, 39, 53, 86, 88, 167, 283, 310, 320, and/or 329 of SEQ ID NO: 3 or amino acid position 102, 129, 139, and/or 148 of SEQ ID NO: 4.
E9. The protease of any one of E6-E8, wherein the protease is from Grifola frondosa.
E10. The protease of any one of E6-E9, wherein the protease comprises an amino acid sequence of SEQ ID NO: 3 or SEQ ID NO: 4.
E11. The protease of any one of E1-E10, wherein the lysine residues of the protease are homogenously chemically modified.
E12. The protease of any one of E1-E11, wherein the lysine residues of the protease are homogenously and completely chemically modified.
E13. The protease of any one of E1-E12, wherein the one or more chemically modified lysine residues are modified with an alkyl moiety, acetyl moiety, amidino moiety, or guanidino moiety.
E14. The protease of E13, wherein the alkyl moiety is selected from the group consisting of a methyl moiety, dimethyl moiety, octanal moiety, and cyclodextrin monoaldehyde moiety.
E15. The protease of E14, wherein the alkyl moiety is a methyl moiety.
E16. The protease of any one of E1-E15, wherein the protease is isolated, recombinant, or synthetic.
E17. The protease of any one of E1-E16, wherein the autolysis resistance of the protease is enhanced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or more as compared to the autolysis resistance of the protease in the absence of the one or more chemically modified lysine residues.
E18. A method of reducing a level of peptide byproducts of protease autolysis in an analytical assay, the method comprising the use of the protease of any one of E1-E17.
E19. The method of E18, wherein the analytical assay is selected from the group consisting of liquid chromatography (LC), LC-mass spectrometry (LC-MS), LC-UV, capillary electrophoresis (CE), gel electrophoresis (GE), and matrix-assisted laser desorption/ionization (MALDI).
E20. The method of E19, wherein the analytical assay is LC-MS.
E21. The method of any one of E18-E20, wherein the method comprises the use of two or more different types of proteases.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A protease comprising one or more chemically modified lysine residues, wherein the protease has enhanced autolysis resistance as compared to the autolysis resistance of the protease in the absence of the one or more chemically modified lysine residues, wherein the protease is a wild-type Endopeptidase Lys-C (Lys-C) protease.

2. The protease of claim 1, wherein the one or more chemically modified lysine residues are at least 1, at least 2, at least 3, at least 4, at least 5, at least 6, at least 7, at least 8, at least 9, at least 10, at least 11, at least 12, at least 13, at least 14, or 15 chemically modified lysine residues.

3. The protease of claim 1, wherein the one or more chemically modified lysine residues are at amino acid position 2, 39, 52, 54, 62, 104, 173, 178, 183, 205, 235, 254, 311, 360, and/or 408 of SEQ ID NO: 1 or amino acid position 30, 49, 106, 155, and/or 203 of SEQ ID NO: 2.

4. The protease of claim 1, wherein the protease is from Achromabacter lyticus.

5. The protease of claim 1, wherein the protease comprises an amino acid sequence of SEQ ID NO: 1 or SEQ ID NO: 2.

6. A protease comprising one or more chemically modified lysine residues, wherein the protease has enhanced autolysis resistance as compared to the autolysis resistance of the protease in the absence of the one or more chemically modified lysine residues, wherein the protease is a wild-type Peptidyl-Lys Metalloendopeptidase (Lys-N) protease.

7. The protease of claim 6, wherein the one or more chemically modified lysine residues are at least 1, at least 2, at least 3, at least 4, at least 5, at least 6, at least 7, at least 8, at least 9, at least 10, or 11 chemically modified lysine residues.

8. The protease of claim 6, wherein the one or more chemically modified lysine residues are at position 25, 39, 53, 86, 88, 167, 283, 310, 320, and/or 329 of SEQ ID NO: 3 or amino acid position 102, 129, 139, and/or 148 of SEQ ID NO: 4.

9. The protease of claim 6, wherein the protease is from Grifola frondosa.

10. The protease of claim 6, wherein the protease comprises an amino acid sequence of SEQ ID NO: 3 or SEQ ID NO: 4.

11. The protease of claim 1, wherein the lysine residues of the protease are homogenously chemically modified.

12. The protease of claim 1, wherein the lysine residues of the protease are homogenously and completely chemically modified.

13. The protease of claim 1, wherein the one or more chemically modified lysine residues are modified with an alkyl moiety, acetyl moiety, amidino moiety, or guanidino moiety.

14. The protease of claim 13, wherein the alkyl moiety is selected from the group consisting of a methyl moiety, dimethyl moiety, octanal moiety, and cyclodextrin monoaldehyde moiety.

15. The protease of claim 14, wherein the alkyl moiety is a methyl moiety.

16. The protease of claim 1, wherein the protease is isolated, recombinant, or synthetic.

17. The protease of claim 1, wherein the autolysis resistance of the protease is enhanced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 120%, at least 140%, at least 160%, at least 180%, at least 200%, at least 250%, at least 300%, at least 350%, at least 400%, at least 450%, at least 500%, at least 600%, at least 700%, at least 800%, at least 900%, at least 1000%, or more as compared to the autolysis resistance of the protease in the absence of the one or more chemically modified lysine residues.

18. A method of reducing a level of peptide byproducts of protease autolysis in an analytical assay, the method comprising the use of the protease of claim 1.

19. The method of claim 18, wherein the analytical assay is selected from the group consisting of liquid chromatography (LC), LC-mass spectrometry (LC-MS), LC-UV, capillary electrophoresis (CE), gel electrophoresis (GE), and matrix-assisted laser desorption/ionization (MALDI).

20. (canceled)

21. The method of claim 18, wherein the method comprises the use of one or more additional protease enzymes.


SEQUENCE LISTING (5 sequences)

SEQ ID NO: 1 - protein, 473 aa, Achromobacter lyticus
MKRICGSLLLLGLSISAALAAPASRPAAFDYANLSSVDKVALRTMPAVDVAKAKAEDLQRDKRGDIPRFALAIDVDMTPQNSGAWEYTADGQFAVWRQRVRSEKALSLNFGFTDYYMPAGGRLLVYPATQAPAGDRGLISQYDASNNNSARQLWTAVVPGAEAVIEAVIPRDKVGEFKLRLTKVNHDYVGFGPLARRLAAASGEKGVSGSCNIDVVCPEGDGRRDIIRAVGAYSKSGTLACTGSLVNNTANDRKMYFLTAHHCGMGTASTAASIVVYWNYQNSTCRAPNTPASGANGDGSMSQTQSGSTVKATYATSDFTLLELNNAANPAFNLFWAGWDRRDQNYPGAIAIHHPNVAEKRISNSTSPTSFVAWGGGAGTTHLNVQWQPSGGVTEPGSSGSPIYSPEKRVLGQLHGGPSSCSATGTNRSDQYGRVFTSWTGGGAAASRLSDWLDPASTGAQFIDGLDSGGGTP

SEQ ID NO: 2 - protein, 268 aa, Achromobacter lyticus
GVSGSCNIDVVCPEGDGRRDIIRAVGAYSKSGTLACTGSLVNNTANDRKMYFLTAHHCGMGTASTAASIVVYWNYQNSTCRAPNTPASGANGDGSMSQTQSGSTVKATYATSDFTLLELNNAANPAFNLFWAGWDRRDQNYPGAIAIHHPNVAEKRISNSTSPTSFVAWGGGAGTTHLNVQWQPSGGVTEPGSSGSPIYSPEKRVLGQLHGGPSSCSATGTNRSDQYGRVFTSWTGGGAAASRLSDWLDPASTGAQFIDGLDSGGGTP

SEQ ID NO: 3 - protein, 348 aa, Grifola frondosa
MFSSVMVALVSLAVAVSANPGLSLKVSGPEAVDGVNNLKVVTTITNTGDETLKLLNDPRGALHTMPTDTFAITNESGETPSFIGVKVKYVPSMAAKSTGENVFAVIAPGQSVNVEHDLSAAYNFTSSGAGTYALEALNVFNYIDPETNEPVEIWADAEAHTTAVSGKLAVVRATPTLTRPVTYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWKAPTTGTDSQAGTLVHESSHFTRNGGTKDYAYGQAAAKSLATMDPDKAVMNADNHEYFSENNPAQS

SEQ ID NO: 4 - protein, 167 aa, Grifola frondosa
TYNGCSSSEQSALAAAASAAQSYVAESLSYLQTHTAATPRYTTWFGSYISSRHSTVLQHYTDMNSNDFSSYSFDCTCTAAGTFAYVYPNRFGTVYLCGAFWKAPTTGTDSQAGTLVHESSHFTRNGGTKDYAYGQAAAKSLATMDPDKAVMNADNHEYFSENNPAQS

SEQ ID NO: 5 - protein, 6 aa, synthetic construct
HHHHHH